(19) United States
(12) Patent Application Publication
(10) Pub. No.: US 2009/0076603 A1
(43) Pub. Date: Mar. 19, 2009

(54) PARTIAL COATING OF INTRAOCULAR LENSES USING ATMOSPHERIC PRESSURE CHEMCIAL VAPOR DEPOSITION

(75) Inventors: Neal H. Avery, Mission Viejo, CA (US); Theophilus Bogaert, Groningen (NL)

Correspondence Address: ADVANCED MEDICAL OPTICS, INC., 1700 E. ST. ANDREW PLACE, SANTA ANA, CA, 92705, US

(73) Assignee: Advanced Medical Optics, Inc., Santa Ana, CA (US)

(21) Appl. No.: 11/857,331

(22) Filed: Sep. 18, 2007

(51) Int. Cl. A61F 2/16; A61L 33/00

(52) U.S. Cl. 623/6.43; 427/2.24; 623/6.38; 623/6.62

(57) ABSTRACT

An atmospheric pressure chemical vapor deposition apparatus has been developed for partially coating acrylic based intraocular lenses. In one embodiment intraocular lenses may be coated with a hydrophilic material comprising silicone dioxide and polyethylene glycol. In another embodiment the haptics may be coated with hydrophilic material, which aids in the post implantation unfolding of the lenses while not diminishing posterior bag adhesion of the hydrophobic surface of the untreated optic portion.

### FIELD OF THE INVENTION

Disclosed are selective coatings of intraocular lenses using atmospheric pressure chemical vapor deposition.

### BACKGROUND

Many present day intraocular lenses are composed of acrylic material. Lenses made of acrylic material are hydrophobic and tend to posses “self tackiness.” This tackiness leads to long unfold times once the lenses are implanted into the eye. Additionally, the haptics associated with an acrylic lens may adhere to the optic and will not release in the eye. In these cases, the acrylic lenses must manually be unfolded once implanted into the eye. Further, the hydrophobicity of particular portions of the lens material, which leads to the tackiness, also may cause increased adhesion to fibers and giant cells within the eye.

Currently, in order to alleviate the problems mentioned above, the entire lens is covered with a hydrophilic coating. A hydrophilic coating allows for more rapid and reliable unfolding of the lens once implanted into the eye, mainly because of the coating of the haptics. Coating the haptics with a hydrophilic coating will result in a more rapid unfolding of the lens upon implantation. Hydrophilic lens surfaces tend to behave better in the eye with respect to interaction with fibers and giant cells.

Despite the excellent behavior of hydrophilic surfaces in the eye, hydrophobic portions of an intraocular lens also have their advantages. One advantage is that hydrophobic surfaces may adhere better to the posterior bag, which will help prevent posterior capsular opacification. Hydrophilic lenses tend not to adhere well to the posterior bag. Therefore, it would be beneficial to coat only portions of the lens with hydrophilic material and leave the remainder untreated. Alternately, making portions of the lens and/or haptics hydrophilic and other portions more hydrophobic than the lens itself are also possible. For example, coating only the haptics with hydrophilic material to aid in unfolding while leaving the lens body untreated to retain posterior bag adhesion might be beneficial.

In addition to the coating of the lens, current commercial methods of coating intraocular lenses are antiquated. Typically, intraocular lenses are coated using vacuum or low pressure chemical vapor deposition assisted by indirect plasma treatment. It is not uncommon for the entire coating process to take 30 minutes or more for a single coat and plasma treatment, not including the time to hermetically seal a section of the lens for partial treatment. The use of an atmospheric pressure chemical vapor deposition method as described herein saves time to coat a lens (requiring about 6 seconds for a single coat and two separate plasma treatments). In addition, the elimination of hermetic seal masking makes the process cheaper and also saves time.

### SUMMARY

Disclosed herein are coated and partially coated intraocular lenses (IOL) and associated methods. Methods include partially coating an IOL using atmospheric pressure chemical vapor deposition wherein the IOL comprises a lens body having an anterior surface, a posterior surface, a circumferential edge and, optionally, at least one support member associated with the lens body. Partially coated lenses using the disclosed methods are also provided

The methods may comprise the steps of (a) providing a first coating material; (b) masking at least a portion of said lens body and optionally said at least one support member to provide a masked IOL; (c) plasma treating the masked IOL to provide a treated masked IOL; (d) applying the first coating material to the treated masked IOL to provide a coated IOL; and, (e) removing the masking from the coated IOL to provide a partially coated IOL. In one embodiment the at least one support member is a haptic.

In one embodiment the coating material is applied by vapor deposition. In another embodiment the masking in Step (b) comprises a physical mask, a chemical mask or combinations thereof The physical mask may be selected from the group consisting of paper, wood, metal or plastic and combinations thereof. In one embodiment the physical mask does not contact the lens surface. In another embodiment the physical mask is created by the opposed lens surface. The chemical mask is selected from the group consisting of a water soluble coating, a lipid soluble coating and combinations thereof. In one embodiment the water soluble coating is a polysaccharide. In another embodiment the lipid soluble coating is selected from the group consisting of wax, adhesive, silicone, methacrylic polymers, and combinations thereof.

In another embodiment at least one of the anterior surface, the posterior surface, the circumferential edge, and the at least one support member is completely masked.

In another embodiment Step (d) is followed by a plasma treating step. In an embodiment this plasma treating step is done under atmospheric conditions.

In another embodiment the treating Step (c) is done under atmospheric conditions. In yet another embodiment the coating Step (d) is done under atmospheric conditions. In yet another embodiment at least one of treating Step (c) and coating Step (d) is done under atmospheric conditions.

In another embodiment the coating material may be selected from the group consisting of silicone dioxide, polyethylene glycol, heparin, lecitine, polyethyleneimine (PEI), poly vinyl pyrrolidone (PVP), fluorine, Polytetrafluoroethylene (PTFE), Polyvinylidene Difluoride (PVDF) and combinations thereof. In yet another embodiment a primer coat is applied between Step (c) and Step (d).

In another embodiment the method is repeated in order to apply at least one additional coating to the partially coated IOL. In another embodiment the method further comprises the steps of: (f) providing a partially coated IOL; (g) providing a second coating material; (h) masking at least a portion of one the anterior surface, the posterior surface, the circumferential edge and the at least one support member to provide a masked partially coated IOL; (i) plasma treating the masked partially coated IOL to provide a treated masked partially coated IOL; (j) applying the second coating material to the treated masked partially coated IOL to provide a differentially coated IOL; and (k) removing the masking from the differentially coated IOL. In one embodiment the second coating material overlaps the first coating material on the differentially coated IOL. In another embodiment the second coating material does not overlap the first coating material on the differentially coated IOL.

In another embodiment a lens body has an anterior surface, a posterior surface, a circumferential edge and, optionally, at least one support member structurally associated with the lens body, wherein at least a portion of one of the anterior surface, the posterior surface, the circumferential edge, and the at least one support member is coated, provided at least one of the anterior surface, the posterior surface, the circumferential edge, and the at least one support member remain uncoated. In one embodiment the at least one support member is a haptic.

In another embodiment the lens body may be made from a material selected from the group consisting of polyhydroxyethylmethylmethacrylate, polymethylmethacrylate, silicone, acrylics, acrylates, poly siloxanes, polyvinyl alcohol, and combinations thereof. In another embodiment the coating is applied by vapor deposition at atmospheric pressure. In yet another embodiment the coating is selected from the group consisting of poly ethylene glycol (PEG), silicone dioxide (SiO₂), heparin, lecitine, polyethyleneimine (PEI), polyvinypyrrolidone (PVP), fluorine, polytetrafluoroethylene (PTFE), polyvinylidene difluoride (PVDF), and combinations thereof.

Several different combinations of the lens body and at least one support member can be selectively coated. In one embodiment only the anterior surface of the lens body is coated. In another embodiment only the posterior surface of the lens body is coated. In yet another embodiment only the circumferential edge of the lens body may be coated. In yet another embodiment only the at least one support member of the lens body is coated. In yet another embodiment only the anterior surface of the at least one support member of the lens body is coated. In yet another embodiment only the posterior surface of the at least one support member of the lens body is coated.

In another embodiment an intraocular lens comprises a lens body having an anterior surface, a posterior surface, a circumferential edge, and optionally at least one support member structurally associated with the lens body, wherein the entire device is coated with one or more layers of silicon dioxide.

### BRIEF DESCRIPTION OF THE DRAWINGS

FIG. 1: The cross section of one apparatus. The apparatus is a device for coating intraocular lenses using atmospheric pressure chemical vapor deposition.

FIG. 2: An intraocular lens.

FIG. 3: An example of a physically masked intraocular lens.

### DETAILED DESCRIPTION

### Definition of Terms

Atmospheric Pressure: As used herein, the term “atmospheric pressure” refers to the open air pressure; as opposed to the pressure in a vacuum or in an enclosed chamber. Atmospheric pressure can vary, but it is typically around 760 torr, but it is highly dependent on the elevation of the manufacturing facility.

Chemical Vapor Deposition: As used herein, the term “chemical vapor deposition,” sometimes referred to herein as “vapor deposition,” describes a process whereby a vaporized material can be used to coat a substrate with a thin film. As described herein, the substrate can be an intraocular lens, the coating can be as described herein, and the deposition can be preformed at atmospheric pressure.

Circumferential Edge: As used herein, the term “circumferential edge,” also known as the optical edge, refers to the area of an intraocular lens where the anterior and posterior surface meet. The circumferential edge surrounds the entire lens except when the haptics or support member(s) are manufactured as a part of the IOL, the circumferential edge surrounds not only the lens, but also the haptics.

Coating: As used herein, the term “coating” (also referred to herein as “coating material”) refers to the material deposited onto the intraocular lens. The coating material used herein can be described by its physical features, for example, its hydrophobicity or hydrophilicity, its anti-reflectivity, or its viscosity. Exemplary coating materials include silicon dioxide (SiO₂), polyethylene glycol (PEG), heparin, lecitine, polyethyleneimine (PEI), poly vinyl pyrrolidone (PVP), fluorine, polytetrafluoroethylene, polyvinylidene difluoride, and combinations thereof.

Differential Coating: As used herein, the term “differential coat” (also referred to herein as “differentially coated”) refers to a second coating that can be different than the first coating. That difference can refer to the orientation of the masking on the optic or the haptics, it can refer to a different coating than has already been applied, or it can refer to a combination of the two.

Intraocular Lens: As used herein, the term “intraocular lens” refers to a lens manufactured to be implanted into the eye typically in place of the natural crystalline lens. Lenses as described herein are generally hydrophobic and can be made of materials including acrylics, acrylates, poly siloxanes, water absorbing acrylates such as polyhydroxyethylmethyacrylate (Poly HEMA), polyvinyl alcohol (PVA), or combinations thereof.

Masking: As used herein, the term “masking” refers to the process of covering a portion of a lens not intended to be coated and the term “mask” refers to the masking substance. A physical mask (or masking) can be a covering that includes substances that do not bind to the lens. Examples of physical masks include, but are not limited to, paper, wood, metal, plastic and combinations thereof. In some embodiments the physical mask can be in contact with portions of the intraocular lens by static electricity. A chemical mask (or masking) can be a covering that includes substances that bind to the lens only during masking. The chemical mask can be weakly bound to the surface and easily removable by rinsing with purified water. Examples of chemical masks include, but are not limited to, wax, adhesive, silicone, polysaccharides, methacrylic polymers, hydroxypropylmethyl cellulose, and combinations thereof.

Support Member: As used herein, the term “support member” refers to an element that helps maintain an intraocular lens in a defined position. An intraocular lens may or may not have a support member associated with it. If the intraocular lens does have a support member associated with it, it will have at least one, but numbers will vary depending on the design, configuration and implant location of the lens. Support members may be made of a continuous material as the optic of the intraocular lens or may be structurally associated with the intraocular lens. A support member can be used to anchor a lens in place, it can be used to rigidly hold a lens in place, or it can act as a spring to keep a lens centered. The term “support member” encompasses haptic(s), which is a well known term to those skilled in the art, and are a common feature(s) found associated with intraocular lenses.

### A System for Atmospheric Pressure Vapor Deposition on Lenses

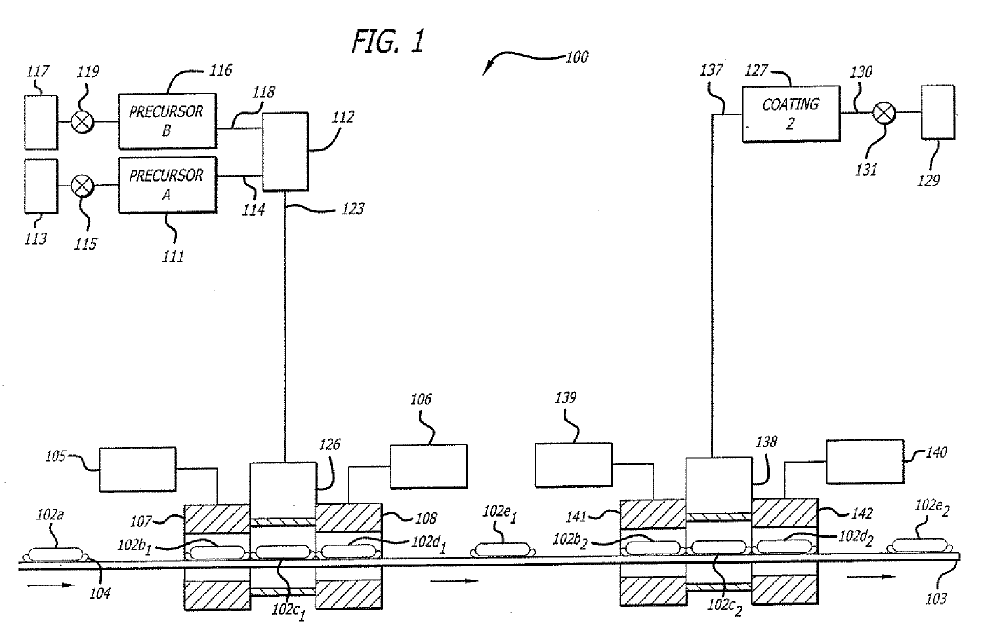

FIG. 1 depicts a schematic of one apparatus 100 for atmospheric pressure chemical vapor deposition of thin coatings on a substrate. In one embodiment apparatus 100 is used to coat intraocular lenses. Substrates to be coated are located on a movable conveyor 103. Conveyor 103 moves the substrate from 102a to 102e₂ (as indicated by the arrows under conveyor 103). Substrate 102 may be moved to various positions on conveyor 103 including, but not limited to position 102a, 102b₁, 102b₂, 102c₁, 102c₂, 102d₁, 102d₂, 102e₁, and 102e₂. Substrate 102 is mounted to a surface treatment fixture 104. Fixture 104 allows the substrate 102 to be processed without damage to substrate 102.

Apparatus 100 has four plasma sources, 105, 106, 139, and 140 connected to plasma treatment heads, 107, 108, 140, and 141. The system also can be configured to use a single plasma source connected to four plasma treatment heads. The application of plasma to the substrate cleans a substrate surface and/or converts a substrate surface to a particular chemical state prior to application of a coating (which enables reaction of coating species and/or catalyst with the surface, to improve adhesion and/or formation of the coating); or may be used to provide species helpful during formation of the coating or modifications of the coating after deposition. The plasma may be generated using microwave, DC, inductive ratio frequency power source, or combinations thereof.

The coating(s) to be applied to substrate 102 may be delivered by at least one of two separate processes. For Coating 1, Precursor A is stored in optionally heated container 111. Precursor A for Coating 1 can be in the form of a solid, liquid, or gas. Coating 1 is directed toward mixing head 112 by a high pressure gas stored in container 113. Container 113 is connected to container 111 and mixing head 112 by gas line 114. The pressure and flow of high pressure gas is controlled by valve 115. The gas can be blown across the top of Precursor A or bubbled through it if Precursor A is a liquid.

In addition, for Coating 1, Precursor B is stored in container 116. A high pressure source of gas is stored in container 117. Precursor A for Coating 1 may be in the form of a solid, liquid or gas. Container 117 is connected to container 116 and mixing head 112 by gas line 118. The pressure and flow of high pressure gas is controlled by valve 119. The gas can be blown across the top of Precursor B or bubbled trough it if Precursor B is a liquid. Liquid precursor can also be metered into the gas stream using atomization or a heated evaporator plate.

Precursor A and Precursor B for Coating 1 may be allowed to react inside mixing head 112 wherein the gas flow becomes turbulent and the two gases mix and react. The temperature of mixing head 112 can be controlled to allow for optimal reaction temperature. Once mixed and reacted, newly formed Coating 1 is transferred to chemical vapor deposition head 126 through Line 123.

Coating 2 may be delivered in the same manner as Coating 1 through a second set of identical hardware; it may be delivered to a chemical vapor deposition head, without being mixed with a precursor in different hardware from Coating 1; Coating 2 may be the same coating material as Coating 1; or it may be delivered as described below.

Coating 2 is stored in optionally heated container 127. Coating 2 may be in the form of a solid, liquid, or gas. Container 129 is connected to container 127 by gas line 130. The pressure and flow of high pressure gas is controlled by valve 131. Liquid precursor can also be metered into the gas stream using atomization or a heated evaporator plate. Coating 2 is delivered to the chemical vapor deposition head 138 through Line 137.

One skilled in the art will appreciate that this procedure is not limited to two coatings, but may be used for several coatings including, but not limited to, primer coatings, catalysts, and cap coats. Several different mixing methods may be used, including, but not limited to, surface catalyzed mixing heads and a catalyst gas added to the mixing head. Several coatings may be mixed before they reach the coating application head to give various mixtures of coatings.

In addition, one skilled in the art will appreciate that using one application head to apply multiple coatings is also possible. For example, in FIG. 1, Line 123 and Line 137 could both direct coatings to chemical vapor deposition head 126. In such a case, position 102c₁ would be where both coatings would be applied to the substrate. If a plasma treatment were to be needed between coatings, the substrate could be moved from 102c₁ to 102b₁ or 102d₁ for a treatment and then back to 102c₁ for a second coating.

In addition, one skilled in the art will appreciate that this entire procedure may be automated. Devices such as, but not limited to, pneumatic valves, solenoid valves, robotic arms, and computer controlled pressure gauges can all be used to aide in the automation process.

### Masking Intraocular Lens for Partial Coating

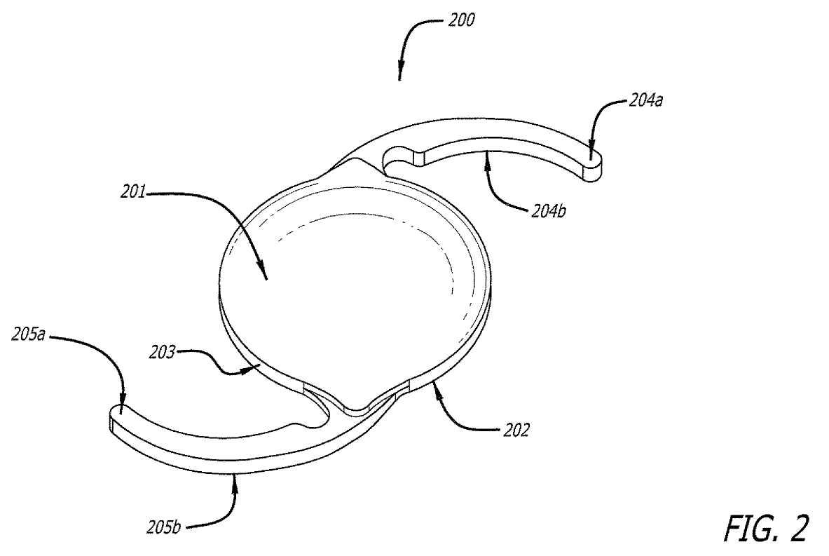

Intraocular lenses may comprise the non-limiting features illustrated in FIG. 2. Intraocular lens 200 has both anterior surface 201 and posterior surface 202. The lens body, and optionally, support members, herein the support members are haptics, can be made from a material selected from, among others, acrylics, acrylates, poly siloxanes, water absorbing acrylates such as Poly HEMA, PVA, and combinations thereof. It will be appreciated that a lens made of these materials likely yield a lens body, and optionally, support members that are hydrophobic. It is also good to note that although the figure depicts the lens with haptics, there are many other support member options and configurations available for intraocular lenses. The lens is concave towards the posterior surface. The posterior surface can be substantially spherical and, after implantation, can reside in contact with the vitreous humor, or posterior bag, in the capsule of the eye. The anterior surface can vary greatly depending on the nature and features of the intraocular lens. Intraocular lens 200 also comprises an optic or circumferential edge 203, a feature that typically leads to reflections seen by the implantee. Intraocular lens 200 further comprises support members 204 and 205, which have anterior surfaces (204a and 205a) and posterior surfaces (204b and 205b). Support members, in this case haptics, may be secured to the lens or may be of a continuous material incorporated into the lens, optionally, on diametrically opposed sides and aid in centering the lens after implantation.

In current commercial methods for the manufacture of acrylic and acrylate based intraocular lenses, the protocol calls for a single or multiple coatings to be applied to the entire hydrophobic lens including the support members. The coating is typically hydrophilic in nature allowing for decreased and more predictable unfold times. However, depending on the application, coating an entire lens with a hydrophilic coating may be less than ideal. As disclosed herein hydrophobic acrylic and acrylate intraocular lenses may be partially coated with hydrophilic coatings and, in some cases, antireflective coatings.

The present coating material can be hydrophilic, hydrophobic, or even have regions of hydrophobicity and hydrophilicity. Exemplary coating materials include silicon dioxide (SiO₂), polyethylene glycol (PEG), heparin, lecitine, polyethyleneimine (PEI), poly vinyl pyrrolidone (PVP), fluorine, polytetrafluoroethylene, polyvinylidene difluoride, and combinations thereof. Multiple coating materials can be applied to an IOL. Each coating applied to an IOL can be of the same material, can each be a different material, or can have several coatings of a single material alternating with one or more coatings of different material.

In order to achieve one or more partial coatings on an intraocular lens coated by atmospheric chemical vapor deposition, the areas of the lens that are to remain uncoated may be masked. Since the treatment is done at atmospheric pressure, a hermetic seal to prevent exposure of the masked area is not required. In vacuum systems, coatings for vapor depositions freely expand into the chamber and, unless the mask is hermetically sealed, the vaporized coating may migrate under the non-sealed mask. In a system at atmospheric pressure this is not an issue.

Masking may be accomplished by physical or chemical means. Physical masking techniques include, but are not limited to, covering with paper, wood, metal, plastic, or combinations thereof. Chemical masking techniques include, but are not limited to, covering with wax, adhesive, silicone, or combinations thereof. A combination of a physical and a chemical mask(s) is also possible.

In addition to simply being able to mask of an area of the lens (support members, optics, anterior, posterior, etc . . . ) it is also possible to mask off a portion of a given area. For example, the outer half of a set of haptics could be coated only by masking the optic and the inner half of the haptics.

A second different partial coating accomplished by masking a portion of a pre-partially coated lens is also possible. Different partial coatings can overlap areas that were previously coated or they can be areas that were not previously coated. Several additional different partial coatings may be applied to a lens. There is theoretically no limit to the number of coatings that can be applied. Examples 17 and 18 provide two specific examples illustrating these two methods.

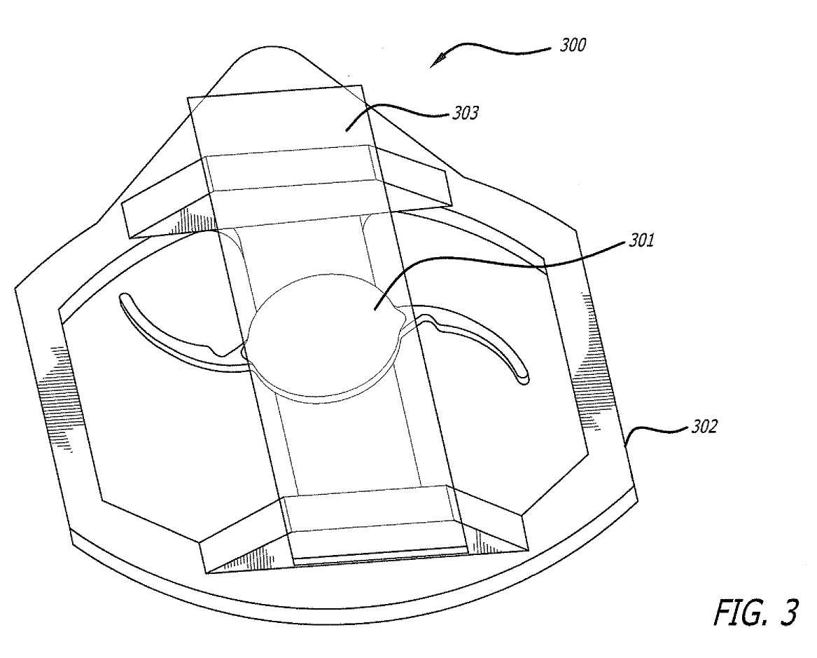

Some non-limiting features of a loaded surface treatment fixture 300 are illustrated in FIG. 3. In one embodiment an intraocular lens 301 is placed on a surface treatment fixture 302. In FIG. 3, for example, a physical mask 303 is used to mask the optic and leave the haptics exposed.

One skilled in the art will appreciate that partial coating configuration options are endless. However, it is important to note the benefits of selectively coating a lens. Since the intraocular lens may be hydrophobic, the lens may adhere to the posterior sack in the eye and help prevent posterior capsular opacification. However, at the same time, moderately hydrophobic lenses are susceptible to interaction with ocular fibers and giant cells and their inherent tackiness may result in slow unfold times after implantation. In some cases, the hydrophobic lens requires physical manipulation to be unfolded after implantation into the eye. Poor unfold times and incomplete unfolding attributed to the tackiness of the haptics. Coating only the haptics with a hydrophilic coating allows for a less tacky surface. More partial coating configurations are described in Examples 11-18.

### EXAMPLES

As described herein intraocular lenses are coated using atmospheric pressure chemical vapor deposition. In the following non-limiting examples, the intraocular lenses are partially coated. However, it should be noted that one skilled in the art will devise, upon reading this description, many ways to coat an entire lens. In addition, the coatings may be replaced, substituted, or combined in accordance with this description and the knowledge of the skilled artisan.

### Example 1

### Providing a Vapor Deposition Coating

The coatings chosen in the present example include silicon dioxide (SiO₂) and polyethylene glycol (PEG). Additional coatings may be used including heparin, lecitine, polyethyleneimine (PEI), poly vinyl pyrrolidone (PVP), fluorine, Polytetrafluoroethylene (PTFE), Polyvinylidene Difluoride (PVDF) and combinations thereof. In addition, a primer coating may be incorporated in certain embodiments.

### Example 2

### Masking

Intraocular lenses similar to that of FIG. 2 may be prepared for a partial atmospheric chemical vapor deposition coating. In this set of examples, the support members associated with the lens are haptics. Lenses typically are prepared in a sterile environment. In order for the lenses to be partially coated, portions of the lens that are to remain uncoated must be masked. In the present example, the optic is chemically masked using wax, covering the lens body and leaving only the haptics exposed to the coatings. Further, in the present example, the anterior surface of the lens body and the haptics will serve as a physical mask for the posterior surface. Therefore, in the present example, only the anterior side of the haptics will be coated.

### Example 3

Once the optics have been properly masked using wax, the lens may be mounted to a surface treatment fixture and placed on the conveyor to be directed into the coating area. The treatment fixture allows the lens to be physically or mechanically rotated or flipped to selectively expose the surfaces of the lens. For example, one side of the lens may be coated then the treatment fixture may be flipped over so the lens may be coated on the opposite side.

### Example 4

### Plasma Pre-Treatment

Once the lens is placed on the conveyor in position 102a (FIG. 1), the conveyor can be progressed such that the fixture/lens unit can be moved to position 102b₁. At position 102b₁, the lens is subjected to plasma treatment via a one inch plasma treatment head. The treatment comprises oxygen plasma. It is necessary to use oxygen plasma, not only to remove contaminants from the lens surface, but to generate hydroxyl functional groups on the lens surface when hydroxyl functional groups are insufficient in number or are not present on the lens surface.

### Example 5

### Applying a Chemical Vapor Deposition Coating 1

After oxygen plasma treatment of the lens at position 102b₁, the lens is moved to position 102c₁ where the coating is applied to the lens by use of a chemical vapor deposition application head. The coating of the present example is silicon dioxide (SiO₂).

Silicon dioxide coating is produced as described above in reference to Coating 1. Silicon tetrachloride is evaporated and carried by inert gas to the mixing head where it is mixed and reacted with water vapor produced by bubbling filtered air through the water. The following reaction takes place in the mixing head.

SiCl₄+H₂O→SiO₂+4HCl

The resulting SiO₂ is directed to the chemical vapor deposition head where it is applied to the plasma treated lens. The treated lens is then moved to 102d₁ where plasma is used to assist in covalently bonding the coating to the IOL substrate. Silicon dioxide reacts with the hydroxyl groups on the surface of the lens creating hydrophilic scaffolding that is covalently bonded to the lens surface.

### Example 6

Upon the completion of the coating and pre and post plasma treatments, the substrate is moved to position 102e₁ where it is up to the operator where the lens is to go next. It can be removed from the conveyor, rotated and moved back to position 102a, flipped and moved back to position 102a, or moved back to position 102a without manipulation for additional coating of the same surface. In the present example, it is progressed to 102b₂ to begin the second coating.

### Example 7

The conveyor can be progressed such that the fixture/lens unit can be moved to position 102b₂. At position 102b₂, the lens is subjected to plasma treatment via a one inch plasma treatment head (similar to that in Example 4).

### Example 8

After oxygen plasma treatment of the lens at position 102b₂, the lens is moved to position 102c₂ where the coating is applied to the lens by use of a chemical vapor deposition application head. The coating of the present example is polyethylene glycol (PEG). PEG may be produced as described above for Coating 2 utilizing a second treatment head. Polyethylene glycol may be evaporated and carried by an inert gas to the second chemical vapor deposition application head where it can be deposited on the lens.

### Example 9

### Plasma Post-treatment

The newly PEG treated lens is moved to position 102d₂ under the plasma treatment head. The plasma treatment helps to assist in covalently bonding the PEG to the SiO₂ layer. The PEG may covalently adhere to the SiO₂ scaffolding already deposited on the lens. The oxygen plasma not only to removes contaminant from the lens surface, but also aids in curing any un-reacted coating material.

### Example 10

After the plasma treatment, the lens may be moved to position 102e₂ where it can be removed from the conveyor, rotated and moved back to position 102a, flipped and moved back to position 102a, or moved back to position 102a without manipulation for additional coating of the same surface. The lens may also be moved to 102e₁ as an alternative to 102a mentioned previously, for a second application of coating 2.

In this set of examples, the lens being coated was subjected to a single application of coating 1 and coating 2. However, in other embodiments, the lens may be subjected to the same process twice for a PEG/SiO2 coating on the anterior side. In addition, once the second set of coatings is completed, the lens/fixture unit may be flipped and the process may be repeated twice on the posterior side of the lens.

### Exemplary Partial Coating Selections

Several partial coating embodiments have been disclosed in the present description. Examples 1-10 describe a single masking embodiment of a single type of intraocular lens; however, it is possible to utilize any of the physical or chemical making techniques described herein for any of the following examples. It may be useful to refer to FIG. 2 in order to generally visualize the masking scenarios.

### Example 11

One partial coating embodiment is described in Example 2. The support members, in this case haptics, would be hydrophilicly coated with SiO₂/PEG (as described in Examples 5 and 8) leaving the masked part (i.e. optic) uncoated. Only the haptics would be hydrophilic and result in an intraocular lens that displayed rapid auto-release characteristics with the added benefit of a hydrophobic optic for better inter-eye adhesion.

### Example 12

Another partial coating embodiment may be to treat the anterior surface of the lens, leaving the posterior side untreated. This would yield a lens with a hydrophilic, SiO₂/PEG coated (as described in Examples 5 and 8), anterior side (both lens body and haptics) and a hydrophobic posterior side. The anterior SiO₂/PEG treatment would allow haptics to auto-release after insertion into the eye while preventing giant cell adhesions on the anterior surface of the optic. The untreated posterior surface likely would adhere better to the posterior bag thereby preventing posterior capsular opacification. It is good to note here that the haptics may be any variation of support member.

### Example 13

Another partial coating embodiment would be to treat the circumferential edge of the lens with an anti-reflective coating. Anti-reflective coatings should be less optically dense than the lens substrate. In this example, the coating could include porous SiO₂. The lens to be partially coated can be an untreated lens or an already partially treated lens (one produced as in example 1-10). The anti-reflective coating would reduce reflections associated with the circumferential edge and hence improve optical performance of the intraocular lens.

### Example 14

Yet another partial coating embodiment would be to treat only the anterior side of the lens, using the anterior surface of the lens as a mask for the posterior side. This procedure would result in a lens with a hydrophobic posterior surface and a hydrophilic anterior surface. In another exemplary embodiment the posterior surface may be coated such that the coefficient of friction is reduced. In this embodiment the posterior surface may be coated with heparin. This would result in an intraocular lens with improved frictional characteristics with an inserter device making the lens easier to implant.

### Example 15

Yet another partial coating embodiment would be to mask the entire optic and associated support member(s) except for the anterior or posterior surface of the support member(s). The benefit, in a case where the support members are haptics, would be haptics that auto release during implantation and adhere to the posterior bag after implantation for improved position stability. Again, haptics could include any applicable support member.

### Example 16

Yet another partial coating embodiment would be to mask the entire anterior optic and support member(s), except for the center of the center of the anterior optic which is left unmasked. The unmasked portion of the anterior optics is coated with a hydrophilic material resulting in a hydrophilic anterior surface with a hydrophobic surface along the periphery for a good sealing of the capsular bag at the anterior surface around the capsularexhis.

### Example 17

A different partial coating can be applied to an already existing partially coated lens to provide a differentially coated lens. In this example, the area to be coated differentially will be an area that was not partially coated during the first partial coating. In this example, a previously partially coated lens from Example 12, wherein the anterior surface of the optic was coated. That lens in now flipped over and the posterior optic is masked and the posterior of the support member(s) are only coated. The resulting lens will be differentially coated on the anterior optic and the posterior surface of the support member(s).

### Example 18

A different partial coating can be applied to an already existing partially coated lens. In this example, the area to be coated differentially will be an area that was partially coated during the first coating. Again in this example, we use a lens form Example 12, wherein the anterior surface of the optic was coated. This lens is now masked over the support members and all of the optic except the center of the optic, leaving a small spot in the center of the optic. The resulting lens will have a coated anterior optic with a spot differentially coated in the center of the optic.

### Example 19

The coatings may also be applied to phakic IOLs. Phakic IOLs are known to those skilled in the art (See e.g. U.S. Pat. No. 7,048,759), but briefly, are correction lenses for implantation in the posterior chamber of the eye between the iris and the intact natural lens. The phakic IOLs comprise a centrally located optical part capable of providing an optical correction and a peripherally located supporting element capable of maintaining said optical part in the central location.

Phakic IOLs may be masked and partially coated using many different combinations of physical and chemical masks, or combinations of physical and chemical masks. An example may be to use the anterior surface of the optic and associated support member as a physical mask for the posterior surface for the optic and the associated support element. The anterior surface of the phakic IOL may then be coated with a hydrophobic material such as polytetrafluoroethylene providing a hydrophobic anterior surface which will prevent adherence of the phakic IOL to the iris.

In another example, the posterior surface of the phakic IOL can be used as a physical mask for the anterior surface of the IOL. The posterior surface phakic IOL may be coated with a hydrophilic coating such as heparin which assures a proper flow of the fluids between the phakic IOL and the anterior lens capsule.

In another example, the two partial coatings for phakic IOLs can be combined, coating the anterior surface of the IOL (including support member) with a hydrophobic material and coating the posterior surface of the IOL (including support member with a hydrophilic material.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles. Other modifications that may be employed are within the scope of the invention. Thus, by way of example, but not of limitation, alternative configurations may be utilized in accordance with the teachings herein. Accordingly, the claims are not limited to that precisely as shown and described.

We claim:

1. A method for partially coating an intraocular lens (IOL) wherein said IOL comprises a lens body having an anterior surface, a posterior surface, a circumferential edge and optionally at least one support member structurally associated with said lens body,
wherein said method comprises:
a) providing a first coating material;
b) masking at least a portion of said lens body and optionally said at least one support member to provide a masked IOL;
c) plasma treating said masked IOL to provide a treated masked IOL;
d) applying said first coating material to said treated masked IOL to provide a coated IOL; and
e) removing said masking from said coated IOL to provide a partially coated IOL.

2. The method according to claim 1 wherein said at least one support member is a haptic.

3. The method according to claim 1 wherein said first coating material is applied by vapor deposition.

4. The method according to claim 1 wherein said masking in step (b) comprises a physical mask, a chemical mask or combinations thereof.

5. The method according to claim 4 wherein said physical mask is selected from the group consisting of paper, wood, metal, plastic and combinations thereof.

6. The method according to claim 4 wherein said physical mask does not contact the lens surface.

7. The method according to claim 4 wherein said physical mask is created by the opposed lens surface.

8. The method according to claim 4 wherein said chemical mask is selected from the group consisting of a water soluble coating, a lipid soluble coating and combinations thereof.

9. The method according to claim 8 wherein said water soluble coating is a polysaccharide.

10. The method according to claim 8 wherein said lipid soluble coating is selected from the group consisting of wax, adhesive, silicone, methacrylic polymers and combinations thereof.

11. The method according to claim 1 wherein at least one of said anterior surface, said posterior surface, said circumferential edge and said at least one support member is completely masked.

12. The method according to claim 1 wherein step (d) is followed by a plasma treatment step.

13. The method according to claim 12 wherein said plasma treatment step is done under atmospheric conditions.

14. The method according to claim 1 wherein at least one of said treating step (c) and said coating step (d) is done under atmospheric conditions.

15. The method according to claim 1 further comprising applying a primer coat between said step (c) and said step (d).

16. The method according to claim 1 wherein said first coating material is selected from the group consisting of silicon dioxide (SiO₂), polyethylene glycol (PEG), heparin, lecitine, polyethyleneimine (PEI), polyvinylpyrrolidone (PVP), fluorine, polytetrafluoroethylene (PTFE), polyvinylidene difluoride (PVDF) and combinations thereof.

17. The method according to claim 1 wherein said method is repeated in order to apply at least one additional coating to said partially coated IOL.

18. The method according to claim 1 wherein said method further comprises the steps of:
f) providing a partially coated IOL;
g) providing a second coating material;
h) masking at least a portion of one of said anterior surface, said posterior surface, said circumferential edge and said at least one support member to provide a masked partially coated IOL;
i) plasma treating said masked partially coated IOL to provide a treated masked partially coated IOL;
j) applying said second coating material to said treated masked partially coated IOL to provide a differentially coated IOL; and
k) removing said masking from said differentially coated IOL.

19. The method according to claim 18, wherein said second coating material overlaps said first coating material on said differentially coated IOL.

20. The method according to claim 18, wherein said second coating material does not overlap said first coating material on said differentially coated IOL.

21. An intraocular lens (IOL) comprising:
a lens body having an anterior surface, a posterior surface, a circumferential edge and optionally at least one support member structurally associated with said lens body;
wherein at least a portion of one of said anterior surface, said posterior surface, said circumferential edge and said at least one support member is coated, provided at least one of said anterior surface, said posterior surface, said circumferential edge and said least one support member remain uncoated.

22. The IOL according to claim 21 wherein said at least one support member is a haptic.

23. The IOL according to claim 21 wherein said coating is applied by vapor deposition at atmospheric pressure.

24. The IOL according to claim 21 wherein said coating is selected from the group consisting of silicon dioxide (SiO₂), polyethylene glycol (PEG), heparin, lecitine, polyethyleneimine (PEI), polyvinylpyrrolidone (PVP), fluorine, polytetrafluoroethylene (PTFE), polyvinylidene difluoride (PVDF) and combinations thereof.

25. An intraocular lens (IOL) comprising:
a lens body having an anterior surface, a posterior surface, a circumferential edge and optionally at least one support member structurally associated with said lens body;
wherein the entire device is coated with one or more layers of silicon dioxide.
